(19) United States
(12) Patent Application Publication
(10) Pub. No.: US 2007/0179448 A1
(43) Pub. Date: Aug. 2, 2007

(54) Dispensing systems

(76) Inventors: Bernard C. B. Lim, Oakville (CA); David G. Matsuura, Encinitas, CA (US); Philip J. Simpson, Escondido, CA (US); Walter Dean Gillespie, San Diego, CA (US)

Correspondence Address: FOLEY & LARDNER LLP, 1530 PAGE MILL ROAD, PALO ALTO, CA, 94304, US

(21) Appl. No.: 10/553,925

(22) PCT Filed: Apr. 23, 2004
(86) PCT No.: PCT/CA04/00604

§ 371 Date: Jul. 19, 2006

(51) Int. Cl. A61M 37/00; A61M 5/00

(52) U.S. Cl. 604/187; 604/131

(57) ABSTRACT

Disclosed herein is a delivery system, comprising dispensing means having an outlet for delivering one or more materials or one or more articles; dispensing control means for controlling the passage of the material or article through the outlet; first identification means operable for recording, emitting, carrying or associating first identity data to identify the dispensing means, or the material or article carried thereby; and permission control means operable to establish a predetermined condition of the dispensing control means when a corresponding predetermined relationship is established between the first identity data and second identity data of an associated entity.

The entire subject matter of each of the following applications is incorporated by reference. The applicant claims priority benefit under Title 35, United States Code, and Section 19(e) of the following applications:
1) U.S. Application Ser. No. 60/464,659, filed Apr. 23, 2003, entitled “DISPENSING SYSTEMS”;
2) U.S. Application Ser. No. 60/523,701, filed Nov. 21, 2003, entitled “MEDICAL MATERIAL HANDLING SYSTEMS”;
3) U.S. Application Ser. No. 60/523,702, filed Nov. 21, 2003, entitled “MEDICAL TREATMENT MANAGEMENT SYSTEMS”

### BACKGROUND OF THE INVENTION

1. Field of the Invention

The present invention relates to the control of materials from dispensing devices, such as for example those which dispense medications and other materials to patients.

2. Description of the Related Art

There has been, in recent years, tremendous changes in the way in which patients are treated. Most social Medicare systems have been scrutinized to improve productivity. Sweeping changes are taking place in the way in which a patient's medial records are tracked, in order to provide a high level of care, while at a reasonable cost. These changes have not occurred, however, without problems. A recent heart lung transplant surgery went horribly wrong because of a relatively minor oversight—a mismatch in the blood type of the donor and recipient patients. This event is overshadowed by numerous accounts of patients being given the wrong medication, which point to the need for improved monitoring and matching of patients with proper and correct medications and/or medical procedures.

It is an object of the present invention to provide a novel materials dispensing system.

### SUMMARY OF THE INVENTION

In one of its aspects, the prevent invention provides a delivery system, comprising:
dispensing means having an outlet for delivering one or more materials or one or more articles;
dispensing control means for controlling the passage of the material or article through the outlet;
first identification means operable for recording, emitting, carrying or associating first identity data to identify the dispensing means, or the material or article carried thereby; and
permission control means operable to establish a predetermined condition of the dispensing control means when a corresponding predetermined relationship is established between the first identity data and second identity data of an associated entity.

In an embodiment, the associated entity is a dispensing recipient, a medical professional or clinician.

In an alternative, the dispensing recipient is a medical patient, an experimental subject and/or a candidate for a treatment or procedure. For example, the dispensing recipient may be mammalian, such as a human being.

In an embodiment, the material or article has beneficial properties to enhance life, to promote health, to cure and/or treat a disease, condition or ailment, to monitor and/or indicate a bodily function or a combination thereof.

In an embodiment, the material or article is useful, amoung others, for IV therapy, implantation, stem cell therapy, oncology therapy, blood transfusion and/or organ transplantation.

In a preferred embodiment, the dispensing means includes a syringe, IV bottle, powder and/or atomized fluid and/or gas inhalant dispenser, implant delivery dispenser, ventilator, syringe pump, intubation tube, gastrointestinal feeding tube or a plurality and/or combination thereof.

It is preferred under certain circumstances that the dispensing control means includes an access means for controlling access to the outlet. In this case, the access means may include a controlled valve member, or a controlled outlet blockage member or both The valve member or outlet blockage member may be normally closed or normally open.

In an embodiment, the valve member is a variable aperture valve member, a proportional valve member or a combination thereof.

In an embodiment, the valve member is a pulse width modulated on-off valve.

In an alternative, the blockage member is a lockable cap member.

In an embodiment, the dispenser means includes a syringe with a plunger portion positioned in a barrel portion, wherein the dispensing control means includes lock means for locking the position of the plunger portion.

The dispensing control means may include a valve means located in the barrel portion or downstream thereof.

In an embodiment, the dispensing control means includes a blockage member located in the barrel portion or downstream thereof.

In an embodiment, the dispenser includes an output channel providing a delivery site, the valve means and/or blockage member being located at, in or near the delivery site.

Preferably, the second identity data identifies the recipient. For example, the second identity data may be embedded in, carried by or emitted by an article carried externally or internally by the recipient.

If desired, the article may include a band or ring to be worn on a leg, arm or neck of the recipient and include an implantable ID chip.

In one case, the permission control means includes a key portion associated with the second identity data.

In this case, the key portion may be located on an article carried externally or internally by the recipient.

The key portion may be operable to engage a complementary key receiving portion to establish the predetermined condition, in which case the key receiving portion may be located on the dispensing means or at some other location. In one example, the article is a wrist band and the dispensing means is a syringe.

In an embodiment, the key-receiving portion includes a key receiving-passage.

In an embodiment, the permission means is operable to expose the key portion to the key-receiving portion.

In this case, the key portion may be movable between a concealed position and the exposed position. Alternatively, the key portion may be stationary relative to the article and the permission means may further comprise a key shroud which is operable between a key-concealing condition and a key-revealing condition.

In an embodiment, the first identification means includes a biometric sensor, an optical character reader, a bar code reader, a magnetic strip reader, or a combination thereof.

In an embodiment, the first identification means includes a signal emitter and/or receiver to emit and/or receive signals in the visible or invisible frequency spectrums.

In another of its aspects, the present invention provides a material dispensing system, comprising:
a material dispenser having material container portion and a material delivery outlet channel portion;
valve means for controlling access to the delivery outlet channel portion;
first identification means operable for recording, emitting, carrying or associating valve identity data to identify the valve means; and
valve control means operable to establish a predetermined condition of the valve means when a corresponding predetermined relationship is established between the valve identity data and identity data of an associated article in a vicinity of the material dispenser.

In an embodiment, the material dispenser is arranged for delivery of materials in the treatment of a patient, wherein the materials are fluids.

In an embodiment, the material container portion includes a syringe, a vial, a catheter and/or an IV bag. In the case of a syringe, the delivery outlet channel portion is downstream of a plunger-containing chamber portion, the valve means being located in the delivery outlet channel portion.

In an embodiment, the delivery outlet channel portion is downstream of a plunger-containing chamber portion, the valve means further comprising a valve housing attachable with and/or separable from the delivery outlet channel portion.

Preferably, the associated article is attachable to or wearable by a patient to receive the fluid materials. In this case, the associated article may be an identity tag attachable to or an article worn by the patient.

Preferably, the system further comprises second identification means operable for recording, emitting, carrying or associating identity data to identify the associated article. The first or second identification means, or both, are arranged to retain the valve identity data or the associated article identity data in electronic, graphical, mechanical or nuclear form. The first or second identification means, or both, are operable to convey the valve identity data or the associated article identity data on a carrier wave. The carrier wave may include radio frequency waves, microwaves or waves or signals of other frequencies or frequency ranges. The valve identity or the associated article identity data may, for example, be resident on the carrier wave by frequency modulation, amplitude modulation, wave superposition or a combination thereof

It is preferred under certain circumstances that the system further comprises comparison means for comparing the valve identity data with the associated article identity data. The comparison means is operable to receive and decode an RFID signal from the valve, the associated article or both. For example, where the containing means includes a syringe, the comparison means may be integrally formed with the syringe. Alternatively, the syringe may be provided with a valve portion downstream of and separable from a chamber portion, in which case the comparison means may be located in the valve portion.

In an embodiment, the comparison means is resident in an intermediate controller module which is operable within signal receiving range of both the valve and the associated article.

In an embodiment, the valve means includes a valve element powered by a power supply portion. The power supply portion includes a power source residing in the power supply portion, a conductive path to an external power source, or an inductive power generating module which is responsive to externally applied radiation, or a combination thereof. For example, the radiation may be of the microwave or radio wave frequency ranges.

In an embodiment the comparison means is operable to open the delivery outlet channel portion when there is a match between the valve identity data and the associated article data. In this case, the comparison means is operable to close the valve means to block access to the delivery outlet portion when there is a mismatch between the valve and the associated article identity data. Similarly, the material dispenser includes a syringe and the power supply portion is integrally formed therewith.

In an alternative, the first and second identification means includes complementary first and second key formations located on, in or near the valve means and the associated article respectively. The first key formation is located on the material dispenser and the second key formation is located on the associated article so that the material dispenser and associated article may positioned so that the first and second key formations be brought into complementary engagement with one another to establish the predetermined relationship.

In yet another of its aspects there is provided a delivery system, comprising:
syringe means having an outlet for delivering fluid materials;
onboard dispensing control means onboard the syringe means for controlling the passage of the fluid materials through the outlet;
first identification means operable for recording, emitting, carrying or associating first identity data to identify the dispensing means, or the material or article carried thereby; and
permission control means operable to communicate with the onboard dispensing control means to establish a predetermined condition of the dispensing control means when a corresponding predetermined relationship is established between the first identity data and second identity data of an associated entity.

Preferably, the associated entity is a dispensing recipient, a medical professional or clinician. The dispensing recipient may be a medical patient, an experimental subject and/or a candidate for a treatment or procedure and the dispensing recipient is mammalian.

It is preferable under certain circumstances that the onboard dispensing control means includes an onboard access means for controlling access to the outlet. The onboard access means includes an onboard controlled valve member, or an onboard controlled outlet blockage member or both. Preferably, the onboard valve member or onboard outlet blockage member is normally closed and may be a variable aperture valve member, a proportional valve member or a combination thereof. In one example, the onboard valve member is a pulse width modulated on-off valve.

In an embodiment, the syringe means includes a plunger portion positioned in a barrel portion, the onboard dispensing control means including lock means for locking the position of the plunger portion. The onboard dispensing control means may include an onboard valve means located in the barrel portion or downstream thereof, or an onboard blockage member located in the barrel portion or downstream thereof.

In an embodiment, the second identity data identifies the recipient and is embedded in, carried by or emitted by an article carried externally or internally by the recipient. The may be include a band or ring to be worn on a leg, arm or neck of the recipient and the article may include includes an RFID tag or chip carrying the second identity data.

### BRIEF DESCRIPTION OF THE DRAWINGS

Several preferred embodiments of the present invention will now be described, by way of example only, with reference to the appended drawings in which:

FIG. 1 is a view of a material dispensing system;

FIG. 2 is a schematic view of one portion of the system of FIG. 1;

FIGS. 3 and 4 are fragmentary schematic views of alternative dispensing systems; and

FIGS. 5 to 21 are views of alternative dispensing systems.

### DESCRIPTION OF THE PREFERRED EMBODIMENTS

Referring to the figures, there is provided a material dispensing system 10 comprising a material dispenser 12 having material container portion 14 and a material delivery outlet channel portion 16. Valve means 18 is provided downstream of the container portion 14 and controls the flow of materials through the delivery outlet channel portion 16. It will be recognized that the material dispenser 12 is in the form of a syringe and is operable to dispense a range of materials normally in the medical treatment of a patient In this case, the delivery outlet channel portion includes an outlet passage 20 extending downstream of the material container portion 14 and upstream of a needle 22 which is provided with a coupling 22a for joining the needle 22 to the outlet passage 20. The coupling may be, for example, a LUER fitting.

The system 10 also includes a wrist band 24 which is wearable by the patient, for reasons to be described.

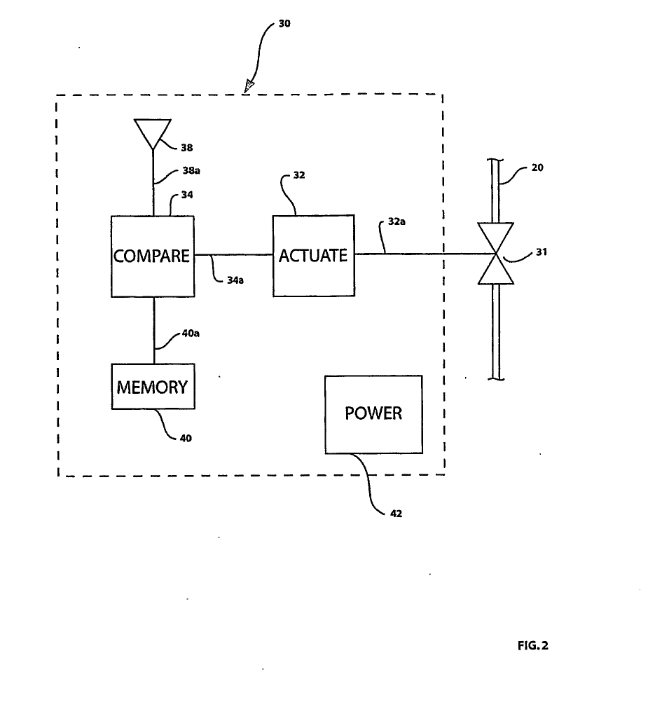

The valve means 18 is controlled by an onboard dispensing control means in the form of a valve controller 30 shown in FIG. 2. The valve means 18, in this case, includes a micro valve 31. The valve 31 has OPEN and CLOSED states which, as their names imply, open and close the delivery output passage 20, under the control of the valve controller 30. The valve controller 30 includes RFID tag or chip and is located within a valve housing 18a. The RFID chip may for example be of the passive type available from Microchip Technology Inc. under serial number MCR 45X, as described in microID™ 13.56 MHz RFID System Design Guide, the entire subject matter of which is incorporated herein by reference.

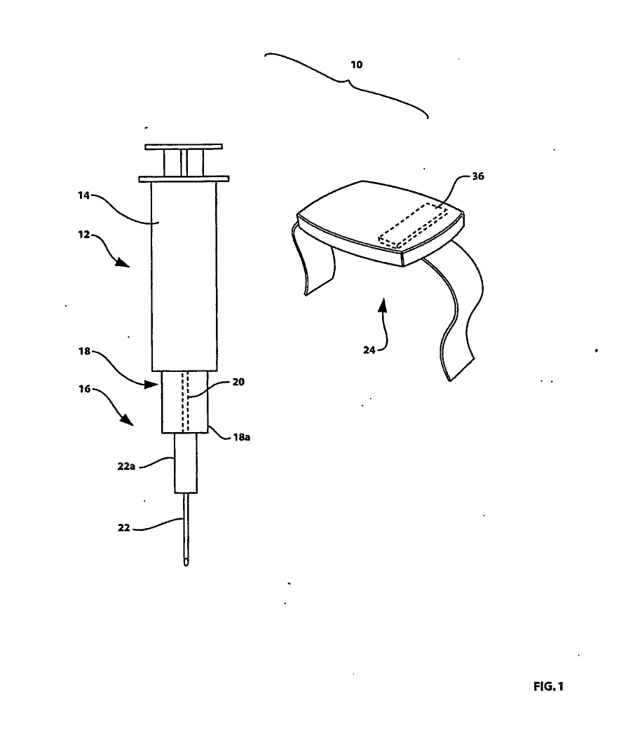

The valve controller 30 includes an actuator module 32 which conveys a change of state signal to the micro valve 31 via conductive path 32a. The actuator module is responsive to a comparator module 34 via conductive path 34a and which establishes a “match” between two identities, a first identity for the valve and/or the materials in the syringe, and a second identity for the patient. As will be described, the valve controller 30 governs the first identity and the wrist band 24 controls the second identity. In this latter case, the wrist band 24 has an RFID tag or chip 36 (shown in FIG. 1) which conveys the second identity on an RF carrier wave to form a second identity bearing signal to be transmitted to the valve controller 30 when they are in transmission range of one another.

The comparator module 34 communicates with an RF transmitter/receiver 38 via conductive path 38a in which to receive the second identity bearing signal from the wrist band 24. The comparator module 34 also communicates with memory module 40 via conductive path 40a to retrieve the first identity stored therein.

The comparator module 34 is operable to open the micro valve 31 and thus the delivery outlet channel portion when there is a match between the valve identity data (i.e. the materials contained in the syringe) and the wrist band 24 (i.e. the patient identified to receive the materials). In this case, the comparator module 34 is operable to convey a signal to the actuator module 32 close the micro valve 31 to block access to the delivery outlet channel portion 16 when there is a mismatch between the first and second identity data. Alternatively, the comparator function may be to maintain the micro valve 31 in a closed position and open it only when the match has been made and thereafter leave the valve open. This arrangement may be particularly suitable for single use dispensers, for instance.

The valve housing 18a in this case is integrally formed with the syringe 12. However, the valve housing 18a may, as an alternative, be made attachable and/or be separable from the delivery outlet channel portion 16 or the syringe 12 or both. For example, the valve may be located in the needle side of the syringe needle coupling. For example, the valve may be located in or adjacent the female stop or lock LUER fittings of the type used on many popular syringes. The valve may be an intermediary unit which is fitted onto a conventional syringe and sealed thereon in some permanent, semi-permanent or temporary means.

The wrist band 24 may, alternatively, be replaced by some other suitable associated article which is attachable to, or wearable by, a patient identified to receive the fluid materials contained in the syringe 12. In this case, the associated article may be an identity tag attachable to or an article worn by the patient.

Thus, the valve controller 30 and the wrist band 24 provide first and second identification means which are operable for recording, emitting, carrying or associating identity data to identify the syringe 12 (and/or its contents) and the wristband 24 (and/or the patient wearing it). The first or second identification means, or both, are arranged to retain their respective identity data in electronic, graphical, mechanical or nuclear form The first or second identification means, or both, are operable, in this case, to convey the first identity data or the second identity data on a carrier wave. The carrier wave may include radio frequency waves, microwaves or waves or signals of other frequencies or frequency ranges. The first identity data or the second identity data may, for example, be resident on the carrier wave by frequency modulation, amplitude modulation, wave superposition or a combination thereof.

The valve controller 30 is powered by a power supply portion 42 which, in this case, includes a power source residing in the power supply portion, such as a battery or some internal power generating module such as solar power generator operating in the presence of solar radiation, or an inductive power generator operating in the presence of microwave or RF radiation. The power supply portion may include a conductive path to an external power source.

If desired, the controller 30 may be resident in the wrist band 24 and thus convey the change of state signal from the actuator module either in a wireless fashion as above described or by way of an electrical or magnetic coupling between the wrist band and the syringe 14. Still further, the controller function may be provided by a controller located on a server-client or a peer-to-peer, wireless or wired network connection with the syringe.

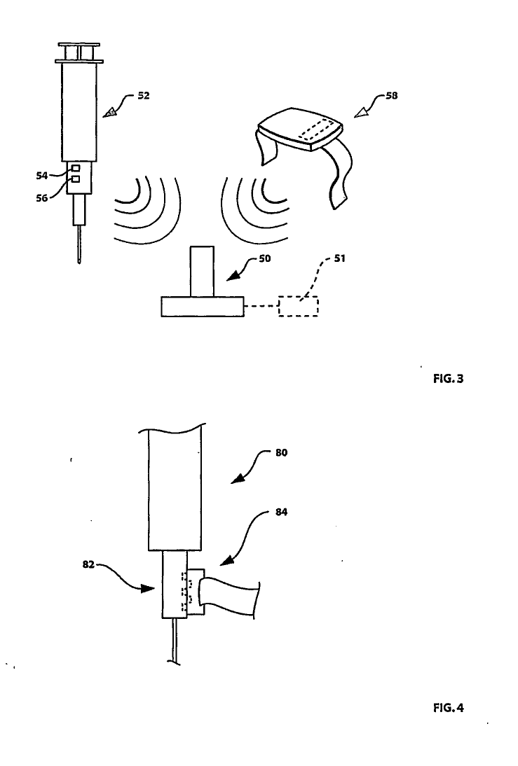

Another embodiment is shown in FIG. 3. In this case, the comparison means is resident in an intermediate monitor portion shown at 50 which is operable within signal receiving range of both a syringe 52 having a valve 54 and a valve actuator 56 and a wrist band 58. In this case, the intermediate monitor portion is operable to convey a signal to the valve actuator 56 to change the state of the valve 54. In this case, the actuator module or the comparator module, or both, may also be contained in the intermediate monitor portion and convey the change of state signal through a suitable data link established between the syringe and the intermediate monitoring portion 50, such as a wired interconnect, a wireless connection and the like.

If desired, the intermediate monitor portion 50 may include a biometric sensor, an optical character reader, a bar code reader, a magnetic strip reader, or a combination thereof, as represented by the block in phantom at 51 in FIG. 3, in this case to scan one or both of the syringe and the wrist band to establish a match. The intermediate monitor portion 50 may also includes include a signal emitter and/or receiver to emit and/or receive signals in the visible or invisible frequency spectrums.

The dispensing control means may not be a valve itself, but rather a valve lock that allows the valve to be opened in a separate step. In this case, a valve and a valve lock may involve two separate functions that are activated dependently or independently. In this case, the valve lock itself may also have functionality to allow it to be remain latched in the unlocked position until the dispensing device is used. Once used, the valve lock may be configured automatically to lock following use, for example when a LUER connector on a needle is removed from a syringe. In this case, the valve may be spring loaded to remain in the closed position unless it is interacted with a LUER connector.

Further, the controlling function for the valve lock may be located either on the dispensing means itself (such as a syringe) or on an intermediate monitor portion (such as a wristband) and then interact with the valve by bringing the wristband and the syringe together to allow them to interact through a wired or wireless connection or through a mechanical connection to allow the wristband to activate the valve or the valve lock Furthermore, the intermediate monitor portion may also house the valve lock controller and, with the wristband and correct syringe located beside it would have authority to unlock it and perform the function of unlocking the syringe.

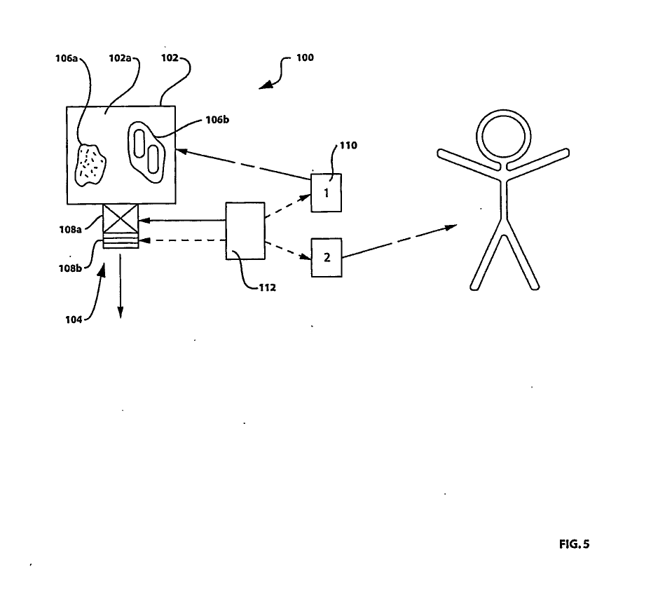

Another embodiment is shown in FIG. 4. In this case, a material dispenser 80 is provided with a valve means 82 and a wrist band 84. In this case, the first and second identification means include complementary first and second key formations located on, in or near the valve means 82 and the wrist band 84 respectively. The first key formation is located on the material dispenser 80 and the second key formation is located on the wrist band 84 so that when the material dispenser 80 and wrist band 84 may be positioned beside one another, the first and second key formations may be brought into complementary engagement with one another to establish the predetermined relationship. In other words, the first and second key formations will nest, mesh or otherwise engage in a manner indicating a match.

Another delivery system is shown, in more general terms, schematically at 100 in FIG. 5 having dispensing means 102 having a chamber 102a and an outlet 104 for delivering one or more materials shown at 106a or one or more articles shown at 106b. A dispensing control means, in this case a valve unit 108a or a lockable outlet blockage member 108b is operable to control the passage of the material or article through the outlet 104. A first identification means is provided at 110 and is operable for recording, emitting, carrying or associating first identity data to identify the dispensing means 102, or the material or article carried thereby. Permission control means 112 is operable to establish a predetermined condition of the dispensing means when a corresponding predetermined relationship is established between the first identity data and second identity data of an associated entity.

In this case, the first condition of the dispensing means may be closed or inoperative position (which is signified by a syringe being in storage) and a second condition may bean open or operative position (which may occur when a match is made between the first and second identity data).

In this case, the valve member or outlet blockage member may be normally closed or normally open. The valve may be of a number of different valve types including a variable aperture valve member, a proportional valve member or a combination thereof. The valve may also be a pulse width modulated on-off valve, while the blockage member may be a lockable cap member or the like.

In this case, the associated entity may be a dispensing recipient, a medical professional or clinician. The dispensing recipient may be a medical patient, an experimental subject and/or a candidate for a treatment or procedure. For example, the dispensing recipient may be mammalian, such as a human being.

The material 106a or article 106b may have beneficial properties to enhance life, to promote health, to cure and/or treat a disease, condition or ailment, to monitor and/or indicate a bodily function or a combination thereof. For example, the material or article may be use useful for such procedures as IV therapy, implantation, stem cell therapy, oncology therapy, blood transfusion and/or organ transplantation, as well as many others.

The material may be in solid, liquid or gaseous form or a combination thereof. Consequently, various dispensing means are contemplated to dispense the material including a syringe, IV bottle, powder and/or atomized fluid and/or gas inhalant dispenser, implant delivery dispenser, ventilator, syringe pump, intubation tube, gastrointestinal feeding tube or a plurality and/or combination thereof, amoung others. The article may be a capsule, for example, to provide or delivery a payload to the recipient, such as a material sample; a local test, monitoring or stabilizing device; a signal device or the like.

In one example, the second identity data identifies the recipient. For example, the second identity data may be embedded in, carried by or emitted by an article carried externally or internally by the recipient. If desired, the article may include a band or ring to be worn on a leg, arm or neck of the recipient and include an implantable ID chip.

In one example, the dispenser means includes a syringe as shown at 120 in FIG. 6, with a plunger portion 122 positioned in a barrel portion 124, wherein the dispensing control means includes lock means 126 for locking the position of the plunger portion. In this case, the dispensing control means may include a valve means located in the barrel portion as shown at 128 or downstream thereof as shown at 130. Alternatively, the dispensing control means includes a blockage member 132 located in the barrel portion or downstream thereof.

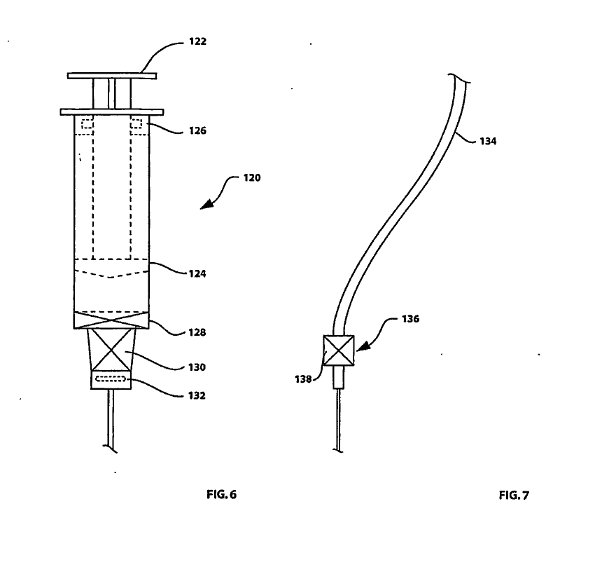

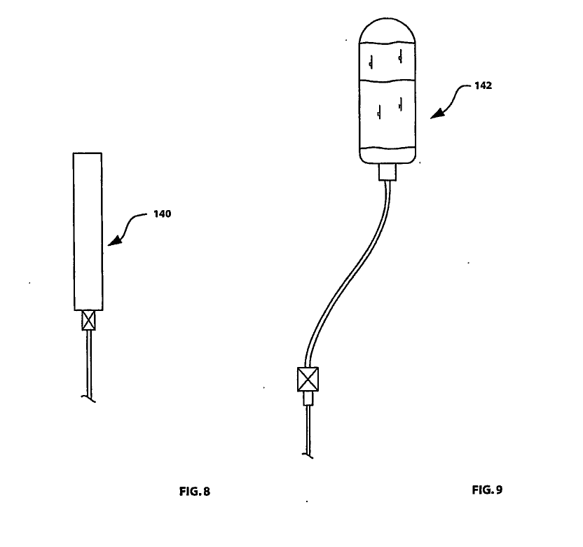

Referring to FIG. 7, the dispenser may include an output channel 134 providing a delivery site 136, the valve means and/or blockage member being located at the delivery site 138. FIGS. 8 and 9 illustrate a vial 140 and an IV bag 142 as still other alternative examples of dispensers applicable in systems described herein.

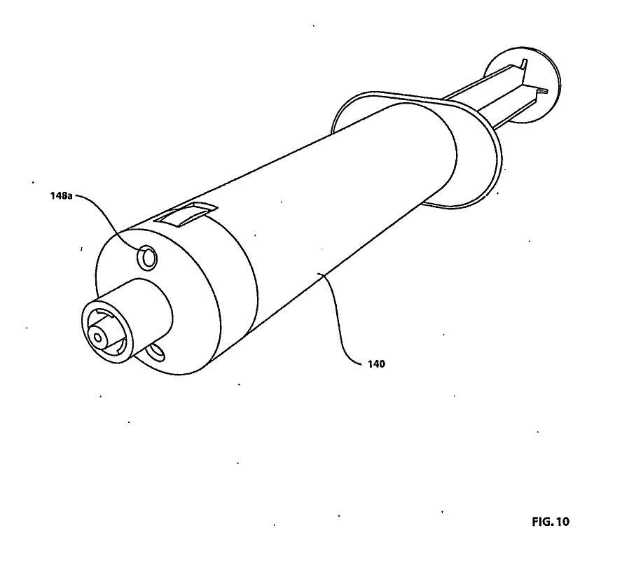

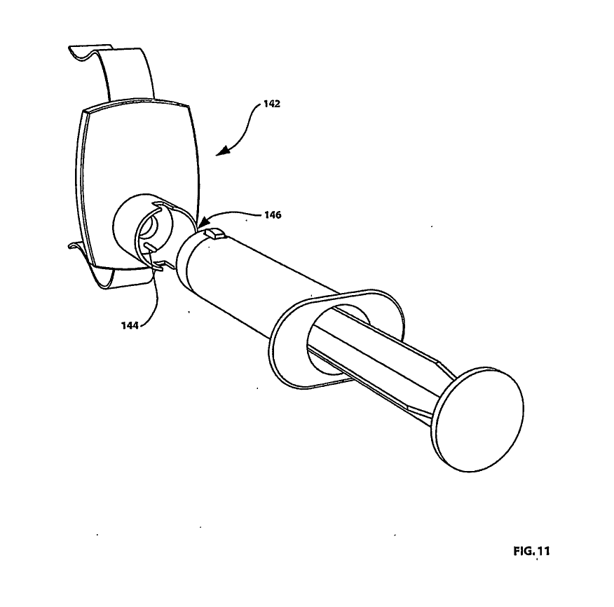

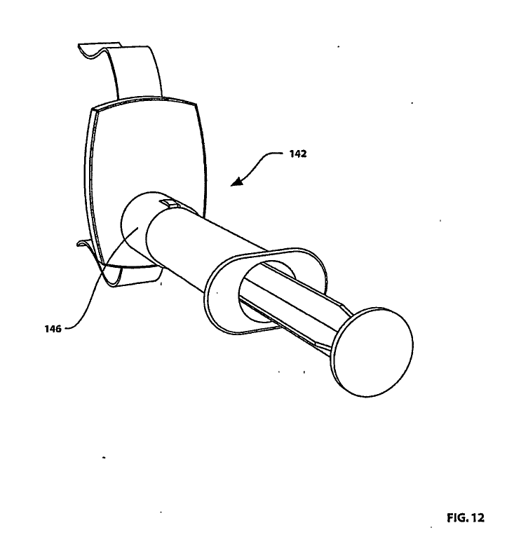

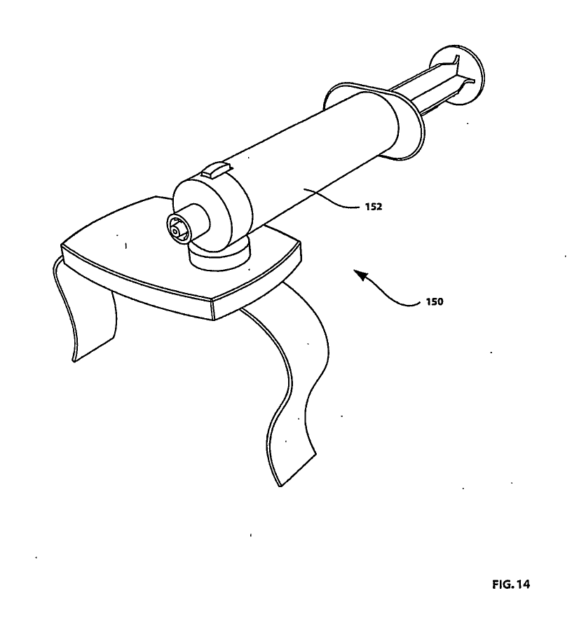

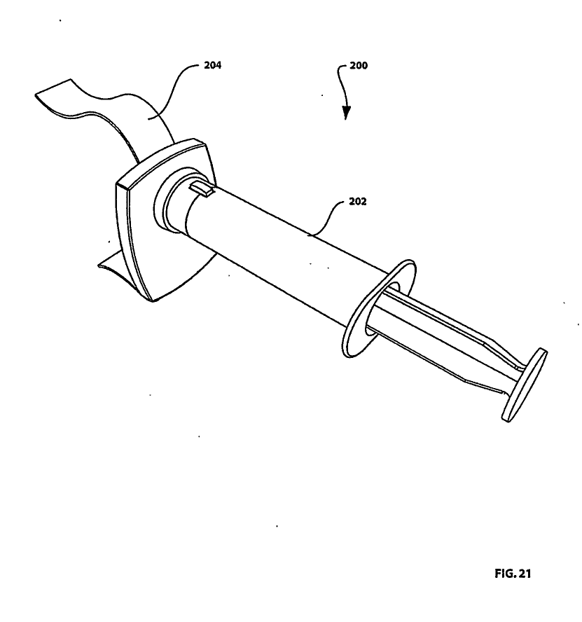

FIGS. 10 to 21 illustrate still other embodiments, all involving the use of a syringe and an identity band. In this case, the permission control means includes a key portion associated with the second identity data. In this case, the key portion is located on the identity band.

FIGS. 10 to 12 show a system having a syringe 140 and a wrist band 142, the latter of which having a key portion 144 in the form of a post extending upwardly from a syringe receiving cavity 146. The key portion is operable to engage a complementary key receiving portion, in the form of a key receiving passage 148a on the syringe 140.

Another system is shown at 150 in FIGS. 13 and 14, having a syringe 152 and a wrist band 154. In this case, the permission means is located on the wrist band at 156 and is operable to expose the key portion to the key-receiving portion. In this case, the key portion is stationary relative to the article and the permission means includes a key shroud 158 which is operable between a key-concealing condition and a key-revealing condition. Alternatively, the key portion may be movable between a concealed position and the exposed position.

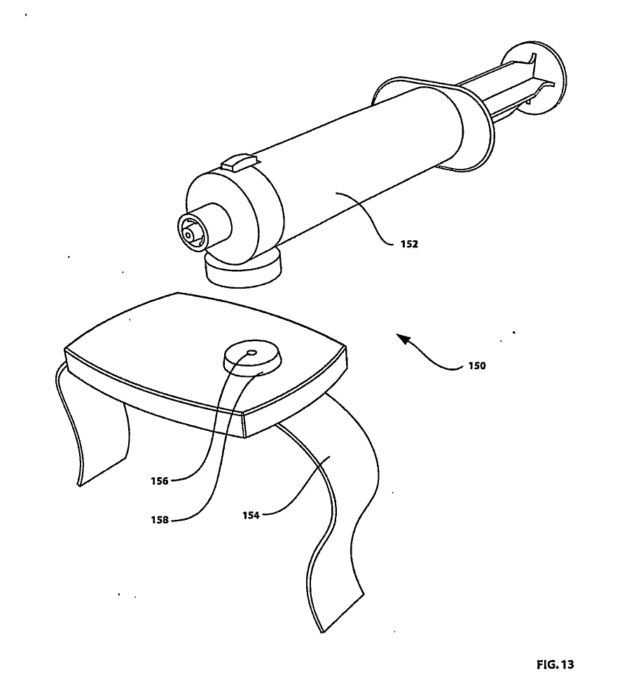

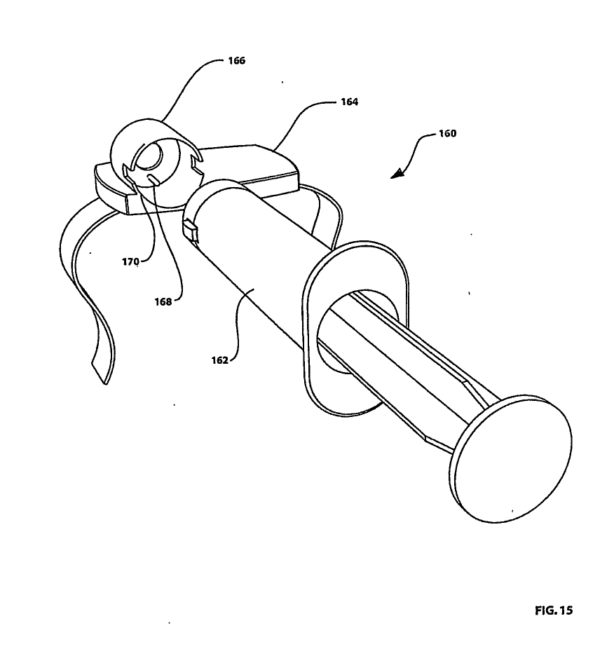

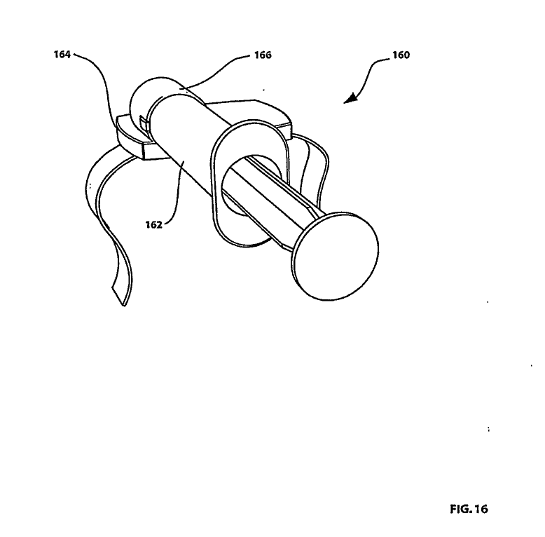

Another system is shown at 160 in FIGS. 15 and 16, having a syringe 162 and a wrist band 164. In this case, the wrist band 164 has a key portion 166 in the form of a post 168 extending outwardly from within a syringe receiving cavity 170. In this case, the syringe receiving cavity 170 is inclined relative to the wrist band 164, in contrast to the upright orientation of the syringe receiving cavity 146 as shown in FIG. 11.

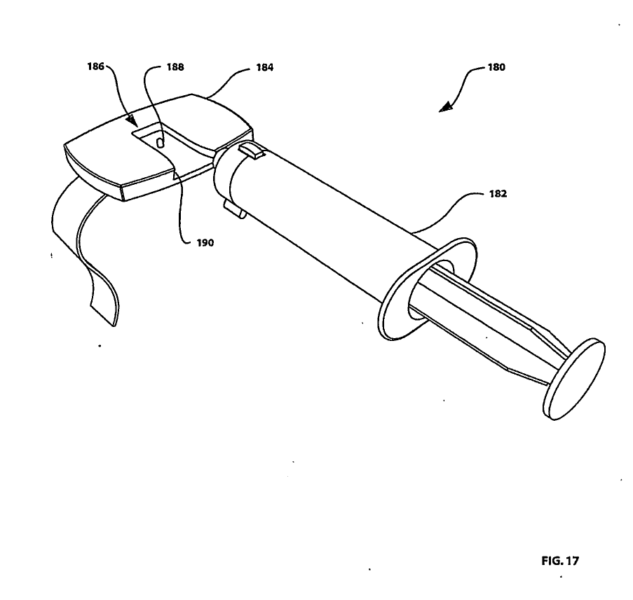

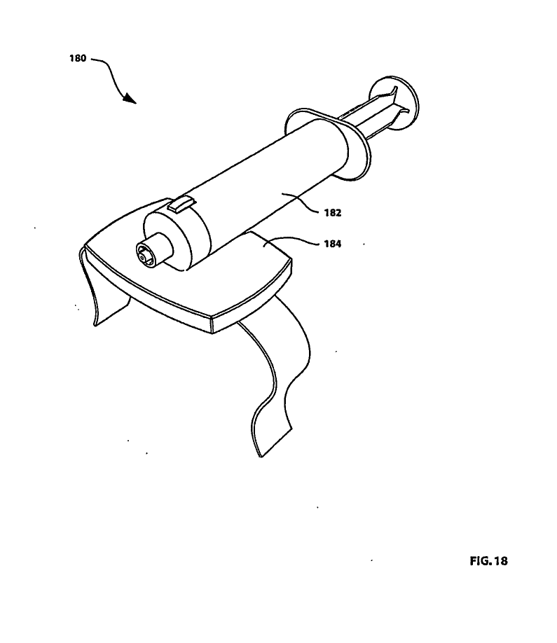

Still another system is shown at 180 in FIGS. 17 and 18, having a syringe 182 and a wrist band 164. In this case, the wrist band 164 has a key portion 186 including a post 188 extending outwardly to interrupt the path of a syringe receiving slot 190.

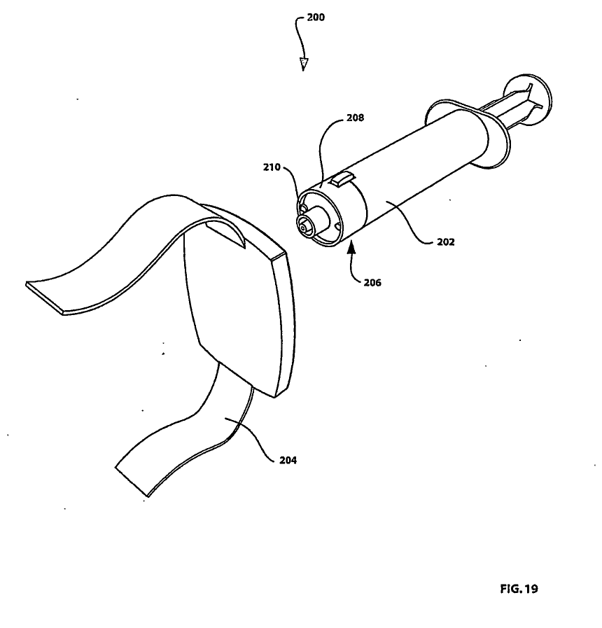

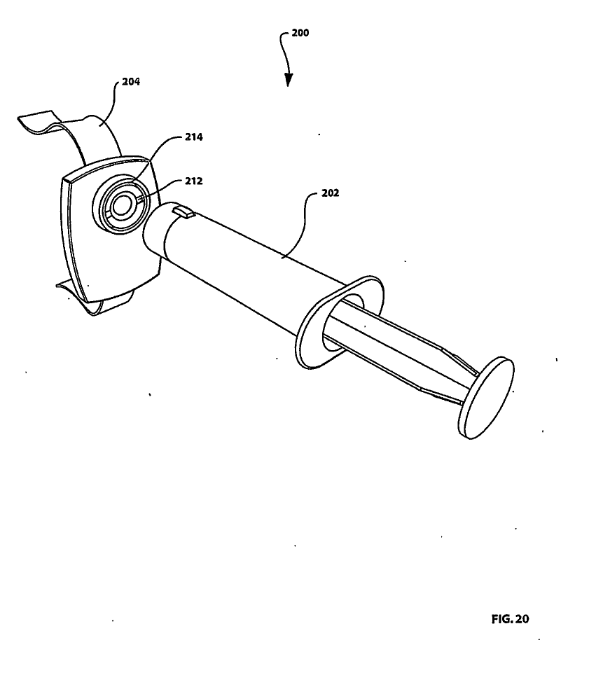

Still another system is shown at 200 in FIGS. 19 through 21 having a syringe 202 and a wrist band 204. In this case, syringe 202 has a key portion 206 including a housing 208 and a pair of posts 210 which, in an operable position, extending outwardly from the housing 208 in order to penetrate a corresponding pair of key receiving passages 212 located within a syringe receiving cavity 214.

If desired, the systems described hereinabove may utilize microvalves, microcontrollers and other micro components known by the acronym MEMS, as disclosed, for example in the technical paper “MEMS-Based Flow Controller for Flow Cytometry”, by Eugen Cabuz, Jay Schwichtenberg, Bob DeMers, Ernie Satren, Aravind Padmanabhan, & Cleo Cabuz, of Honeywell Intl. 12001 State Highway 55, Plymouth, Minn. 55441, the entire contents of which is incorporated herein by reference.

While the present invention has been described for what are presently considered the preferred embodiments, the invention is not so limited. To the contrary, the invention is intended to cover various modifications and equivalent arrangements included within the spirit and scope of the appended claims. The scope of the following claims is to be accorded the broadest interpretation so as to encompass all such modifications and equivalent structures and functions.

1-49. (canceled)

50. A delivery system, comprising:
a dispenser having an outlet for delivering one or more materials;
a dispenser control for controlling the passage of the material through the outlet;
a first identifier having first identity data associated with the dispenser or the material carried thereby, the first identifier operable in a data recording mode or a data emitting mode;
a second identifier means having second identity data associated with an entity, the entity being associated with the dispenser, the second identifier operable in a data recording mode or a data emitting mode; and
a permission controller operable to establish a predetermined condition of the dispenser control when a corresponding predetermined relationship is established between the first identity data and the second identity data.

51. A system as defined in claim 50 wherein the entity is a dispensing recipient, a medical professional or a clinician.

52. A system as defined in claim 51 wherein the entity is a dispensing recipient and the second identity data is embedded in, carried by or emitted by an article carried externally or internally by the recipient.

53. A system as defined in claim 50 wherein the dispenser control includes an access controller for controlling access to the outlet.

54. A system as defined in claim 53 wherein the access controller includes a valve, an outlet blockage member, or both.

55. A system as defined in claim 54 wherein the valve or outlet blockage member is operable between an open position and a closed position, and is normally closed.

56. A system as defined in claim 55 wherein the access controller is a valve comprising a variable aperture valve member, a controlled valve member, a proportional valve member or a combination thereof.

57. A system as defined in 56 wherein the valve is a pulse width modulated on-off valve.

58. A system as defined in claim 54 wherein the access controller includes an outlet blockage member comprising a lockable cap member.

59. A system as defined in claim 50 wherein the first or second identifier, or both, are arranged to retain the first identity data or the second identity data in electronic, graphical, mechanical or nuclear form.

60. A system as defined in claim 59 wherein the first or second identifier, or both, are operable to convey the first identity data or the second identity data on a carrier wave.

61. A system as defined in claim 60 wherein the carrier wave includes radio frequency waves, microwaves or waves or signals of other frequencies or frequency ranges.

62. A system as defined in claim 60 wherein the first identity or the second identity data is resident on the carrier wave by frequency modulation, amplitude modulation, wave superposition or a combination thereof.

63. A system as defined in claim 50 or claim 60 wherein the permission controller includes a comparator for comparing the first identity data with the second identity data.

64. A system as defined in claim 63 wherein the comparator is operable to receive and decode an RFID signal from the dispenser, the entity or both.

65. A system as defined in claim 50 wherein the first identifier is operable to convey the first identity data in a form detectable by a biometric sensor, an optical character reader, a magnetic strip reader, an RFID reader or a combination thereof.

66. A system as defined in claim 50 wherein the first identifier includes a signal emitter and/or receiver to emit and/or receive signals in the visible or invisible frequency spectrums.

67. A system as defined in any of claims 51 or 52 wherein the second identifier comprises a band or ring to be worn on a leg, arm or neck of the recipient.

68. A system as defined in claim 67 wherein the second identifier includes an identification chip such as an RFID tag associated with the second identity data.

69. A system as defined in claim 53 wherein the access controller is a valve and the first identity data includes valve identity data to identify the valve; and the second identification data includes article identity data to identify an article associated with the entity; the permission controller being operable to open the outlet when there is a match between the valve identity data and the article identity data.

70. A system as defined in claim 69 wherein the permission controller is operable to close the valve to block access to the outlet when there is a mismatch between the valve identity data and the article identity data.

71. A system as defined in claim 69 or 70 wherein the permission controller is resident in an intermediate controller module which is operable within signal receiving range of the valve and the article.

72. A system as defined in claim 69 or 70 wherein the permission controller is integrally formed within the dispenser.

73. A system as defined in claim 54 wherein the access controller comprises a valve powered by a power supply.

74. A system as defined in claim 73 wherein the power supply includes a power source residing in the power supply, a conductive path to an external power source, or an inductive power-generating module which is responsive to externally applied radiation, or a combination thereof.

75. A system as defined in claim 74 wherein the power supply portion is integral within the dispenser.

76. A system as defined in claim 74 wherein the power supply is an inductive power generating module, and the externally applied radiation is within the microwave or radio wave frequency ranges.

77. A system as defined in claim 50 wherein the permission controller comprises a key associated with the second identity data.

78. A system as defined in claim 77 wherein the key is located on an article carried externally or internally by the entity.

79. A system as defined in claim 77 wherein the key is operable to engage a complementary key receiver to establish the predetermined condition.

80. A system as defined in claim 79 wherein the key receiver is located on the dispenser.

81. A system as defined in claim 79 or 80 wherein the key receiver includes a key-receiving passage.

82. A system as defined in claim 81 wherein the permission controller is operable to expose the key to the key receiver.

83. A system as defined in claim 82 wherein the key is movable between a concealed position and an exposed position.

84. A system as defined in claim 82 wherein the key portion is stationary relative to the article and the controller further comprises a key shroud which is operable between a key-concealing condition and a key-revealing condition.

85. A system as defined in claim 52 wherein the first and second identifiers include complementary first and second key formations located on, in or near the valve and the article respectively.

86. A system as defined in claim 85 wherein the first key formation is located on the dispenser and the second key formation is located on the article so that the dispenser and the article may positioned so that the first and second key formations be brought into complementary engagement with one another to establish the predetermined relationship.

87. A system as defined in claim 50 wherein the dispenser comprises at least one member selected from the group consisting of syringe, IV bottle, powder dispenser, atomized fluid dispenser, gas inhalant dispenser, implant delivery dispenser, ventilator, syringe pump, intubation tube and a gastrointestinal feeding tube.

88. A system as defined in claim 87 wherein the dispenser is a syringe having a barrel portion, and plunger portion, the plunger portion positioned in the barrel portion.

89. The system as defined in claim 88 wherein the dispenser control is a lock for locking the position of the plunger.

90. A system as defined in claim 88 wherein the syringe has a valve downstream of and separable from the barrel, and wherein the permission controller includes a comparator for comparing the first identity data with the second identity data, the comparator being located at the valve.

91. A system as defined in claim 88 wherein the dispenser control includes a valve located in the barrel portion or downstream thereof.

92. A system as defined in claim 88 wherein the dispenser control includes a blockage member located in the barrel or downstream thereof.

93. A system as defined in claim 88 wherein the outlet is downstream of the barrel and the dispenser control includes a valve in a valve housing attachable with and separable from the outlet.

94. A system as defined in claim 50 wherein the entity is a dispensing recipient selected from a medical patient, an experimental subject and a candidate for a treatment or procedure.

95. A system as defined in claim 94 wherein the dispensing recipient is mammalian.

96. A system as defined in claim 95 wherein the dispensing recipient is a human being.

97. A system as defined in claim 94 wherein the material has beneficial properties to enhance life, to promote health, to cure and/or treat a disease, condition or ailment, to monitor and/or indicate a bodily function or a combination thereof.

98. A system as defined in claim 94 wherein the material is useful for IV therapy, implantation, stem cell therapy, oncology therapy, blood transfusion and/or organ transplantation.
